(19) United States
(12) Patent Application Publication
(10) Pub. No.: US 2022/0346706 A1
(43) Pub. Date: Nov. 3, 2022

(54) Systems and Methods Related to Compliance Monitoring

(71) Applicants: Mohit Gilotra, Ellicott City, MD (US); Anshum Sood, Chicago, IL (US)

(72) Inventors: Mohit Gilotra, Ellicott City, MD (US); Anshum Sood, Chicago, IL (US)

(21) Appl. No.: 17/734,937

(22) Filed: May 2, 2022

Related U.S. Application Data
(60) Provisional application No. 63/181,984, filed on Apr. 30, 2021.

(51) Int. Cl. A61B 5/00; A61F 5/37

(52) U.S. Cl. CPC A61B 5/4833; A61F 5/3738

(57) ABSTRACT

Systems and methods for recording data representative of, and reviewing and analyzing such data for, compliance by patients with prescribed use regimens of rehabilitation devices, such as a sling, are disclosed. Such devices comprise one or more sensors, a power source, and a communication system. The sensors track data indicative of the actual wear or use time of the rehabilitation device, which may be used to monitor compliance with physician recommended wear times. Adjustments may then be made to the prescribed use regimen, or physical adjustments may be made directly to the rehabilitation device.

### RELATED APPLICATIONS

This application claims the benefit of U.S. Provisional Patent Application Ser. No. 63/181,984, filed 30 Apr. 2021, and entitled “Compliance Monitoring Device,” which is incorporated herein by reference in its entirety.

### BACKGROUND

Adherence to medical treatment can be defined as the extent to which patients follow, or comply with, treatment protocols prescribed by their physicians or other advisor (e.g., physical therapist). Adherence rates are conventionally thought to be higher for acute conditions than for chronic illnesses. Although adherence rates are difficult to measure, compliance should be encouraged as higher adherence rates usually correlate to better clinical outcomes. This trend is commonly noted in medication regimens for diseases such as cancer, hypertension, schizophrenia, and many others.

The World Health Organization has identified poor adherence as one of the major causes of failure to recover from long-term illnesses. It has been reported that poor adherence might be connected to worse functional outcomes in patients with rotator cuff repairs, and there are indicators of varying effects of social and demographic factors with patients' self-reported adherence and functional outcomes by adherence measurement questionnaire in the rotator cuff repair population. Generally, compliance as reported by a patient is unreliable.

An accurate measurement of compliance (e.g., recommended shoulder sling wear or other rehab device usage) is an important research tool. Patients may be instructed to wear a sling after rotator cuff surgery to avoid excessive strain on the repair, which can possibly decrease the incidence of re-tear. However, prolonged shoulder sling immobilization has been linked to shoulder stiffness. Slings are often prescribed for consistent use for a 6- to 8-week period, but no study to date has measured optimal sling wear time after shoulder surgery. There is no definitive link between a particular, measurable condition to be rehabilitated (e.g., torn rotator cuff) and a particular prescribed rehabilitative use of a device. Studies have attempted to correlate functional outcomes with prescribed rehabilitation regimens, but more is needed in the art to clarify the ambiguity surrounding appropriate postoperative rehabilitation compliance (e.g., sling-wearing time).

As stated, in orthopaedics, immobilization may be prescribed either postoperatively or as the primary intervention to manage idiopathic conditions such as scoliosis or clubfoot. After rotator cuff repair, for instance, a sling is commonly used to prevent strain at a tendon repair site because excessive strain occurring during the prescribed immobilization period can lead to failure of the repair. It is unknown to what extent patient adherence to the rehabilitation protocol affects functional outcome. One prior study relied on patient surveys to evaluate adherence to the postoperative protocol; however, methods of collecting data used in such study are likely inaccurate. While previous studies have used temperature sensor technology to objectively measure brace adherence found that self-reporting of adherence indicates a higher than the temperature-recorded adherence rates. In those studies, the temperature sensors used in braces, secured to and supported by the braced body part and were in close contact with patients' bodies. However, shoulder slings and other orthosis may be generally looser (e.g., not tightly secured to the body part) and other treatment implements (e.g., foam rollers, yoga blocks, etc.) may not be carried by or secured to a human body at all. There is no known device, system or method for utilizing temperature variations to determine positional (or implement use) compliance with respect to such loose devices (e.g., slings) or not worn (e.g., treatment implements) devices. Therefore, the art of measuring, reporting, and encouraging compliance with a positional treatment regimen, especially with respect to clothing, devices, or implements that may not be tightly secured to the respective body part.

### SUMMARY OF THE INVENTION

The present invention provides improved devices, systems and methods for measuring, reporting, and encouraging compliance with a positional treatment regimen, especially with respect to clothing, devices, or implements that may not be tightly secured to the respective body part.

According to an aspect of an embodiment according to the present invention, a device includes a substrate in a form suitable for providing a rehabilitation of a portion of a human body, such as a shoulder sling, a roller device, a yoga block, a yoga or stretching strap, etc. The device further includes one or more sensors capable of detecting a representation of a use of the substrate, nonvolatile memory for storing detected representations as electronic data, a communication system capable of transmitting the electronic data to an electronic device, and a power source suitable for powering the one or more sensors, the nonvolatile memory, and the communication system.

According to another aspect of an embodiment according to the present invention, where the device is a shoulder sling, each of the one or more sensors is placed at a location selected from the group consisting of: an inner aspect of a bolster to be positioned between the shoulder sling and a human abdomen, a first inner aspect of the shoulder sling to be positioned proximate a human medial elbow, and a second inner aspect of the shoulder sling to be positioned proximate a human carpometacarpal joint palmar surface.

According to still another aspect of an embodiment according to the present invention, the nonvolatile memory receives and maintains the electronic data directly or indirectly from the one or more sensors, where the detected representations are at least one selected from the group consisting of: temperature measurement, capacitance, light level, human pulse rate, and human pulse-oxygenation level. The communication system is preferably capable of communicating (through wired or wireless communication) the data to an external storage medium disposed in the electronic device, either transmitting automatically and/or in response to a request from the electronic device.

According to yet another aspect of an embodiment according to the present invention, the electronic device is selected from the group consisting of a personal computer, a mobile phone, a tablet computer.

According to a further aspect of an embodiment according to the present invention, the one or more sensors are capable of detecting a representation of a use of the substrate at a programmable sample rate.

According to a still further aspect of an embodiment according to the present invention, the rehabilitation device comprises a brace.

According to an aspect of an embodiment of a method according to the present invention, the method includes the steps of providing a rehabilitation device (e.g., a sling or brace or other orthoses or therapeutic device) comprising one or more sensors, the sensor(s) capable of logging data into nonvolatile memory, the data being representative time that the rehabilitation device is used by a human. The logged data from the one or more sensors is preferably compared to at least one of data manually recorded by the human and first prescriptive use regimen data sourced from someone other than the human, and the logged data is preferably archived.

According to another aspect of an embodiment of a method according to the present invention, the rehabilitation device further comprises a communication system and wherein the tracking step further comprises communicating the actual wearing time data to the communication system.

According to still another aspect of an embodiment of a method according to the present invention, the method further includes the step of transmitting the data to an external storage medium.

According to yet another aspect of an embodiment of a method according to the present invention, the method further includes the step of physically altering (e.g., adjusting or exchanging) the rehabilitation device.

According to yet another aspect of an embodiment of a method according to the present invention, the one or more sensors comprise at least one of the following: temperature sensor, capacitance sensor, pulse-oxygenation sensor, heart rate sensor, or blood pressure sensor.

According to a further aspect of an embodiment of a method according to the present invention, the method further including the step of generating a second prescriptive use regimen data that is different than the first prescriptive use regimen data.

### BRIEF DESCRIPTION OF THE DRAWINGS

FIG. 1 is a partial perspective view of an embodiment of a rehabilitation device according to the present invention disposed on a human body.

FIGS. 2a-2c show diagrammatic representations of rehabilitation device embodiments with predetermined sensor locations.

FIG. 3 is a graph depicting ambient experimental temperature data collection.

FIG. 4a is a flow chart of a first optional method of determining a wear start time according to the present invention.

FIG. 4b is a flow chart of a second optional method of determining a wear start time according to the present invention.

FIG. 5 is a flow chart of a method of determining a wear end time according to the present invention.

FIG. 6 is a graph depicting temperature data collection during a typical session wearing or using a rehabilitation device according to the present invention.

FIG. 7 is a graph depicting temperature data collected from actual wear or use time of a rehabilitation device according to the present invention for a user.

### DETAILED DESCRIPTION

Although the disclosure hereof enables those skilled in the art to practice the invention, the embodiments described merely exemplify the invention which may be embodied in other ways. While the preferred embodiment has been described, the details may be changed without departing from the invention, which is defined by the claims.

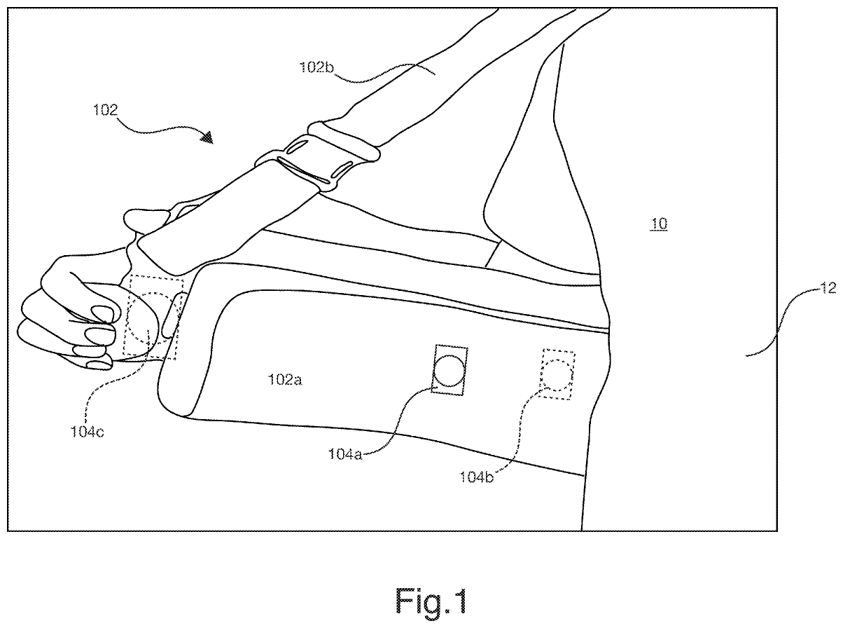

Provided herein are devices, systems and methods configured to more accurately monitor and measure compliance with a predetermined use regimen of a rehabilitation device. Referring to FIG. 1, a rehabilitation device 100 including a substrate in the form of a sling 102 (which may include bolster 102a) can be seen supported on a human body 10, such as with a shoulder strap 102b. The device 100 includes one or more sensors 104 (e.g., temperature sensors). Each sensor 104 preferably includes an onboard, optionally rechargeable power source and nonvolatile memory in which to store measured data points at a fixed, predetermined and/or programmable rate (e.g. data collected and stored approximately once per second, per plurality of seconds (e.g., once per thirty seconds), per minute, per plurality of minutes (e.g., once per thirty minutes), per hour, or per plurality of hours (e.g., once per twelve hours), or per combination thereof (e.g., once per two hours, one minute and twenty-three seconds)). The sensors 104 preferably log respective sensed data (e.g., temperature, light, capacitance, physiological) and store that data in their nonvolatile memories, the data being retrievable by or transmittable to a computing device, such as a personal computer (e.g. running a MacOS or Windows operating system) or a mobile smartphone (e.g., running iOS or Android operating system). A preferred sensor is available from Onset Computer Corporation, and systems and methods for transferring sensor data can be found in U.S. Pat. No. 8,860,569, to Hruska et al., which is incorporated by reference herein in its entirety. Accordingly, each sensor 104 preferably includes a communications interface, such as a Bluetooth Low Energy (BLE) wireless interface or USB wired interface, and a suitable a conventional, optionally rechargeable, power source (e.g., a battery, such as a lithium button cell).

While a preferred sensor may be a temperature sensor, the sensor may additionally or alternatively be or include a capacitance sensor, light (or lack thereof, as in darkness) sensor, or other physiological sensors such as pulse, pulse-oxygenation, heart rate, blood pressure, or the like. The communication system (packaged with the sensor in the Onset devices) is configured to receive the signal from each sensor 104. In one embodiment, the computing device is configured to store and/or transmit data. For example, the data may be stored and transmitted to an external storage or reader (e.g., a cloud network, other personal computer, wearable device, smartphone, or the like) at regular intervals to reduce power consumption. The communication system may also be configured to transmit data only when requested by a monitor, external storage, or reader to further reduce power consumption and complexity. In some embodiments, the communication system is wireless according to typical standards (e.g., WIFI, Bluetooth, etc.) or wired. The power source, such as a battery or solar panel, provides power to the sensor and/or communication system.

Each sensor 104 is positioned on or within a rehabilitation device to sufficiently measure temperature change over a predetermined period as a proxy for use of the device, pursuant to the algorithm described below.

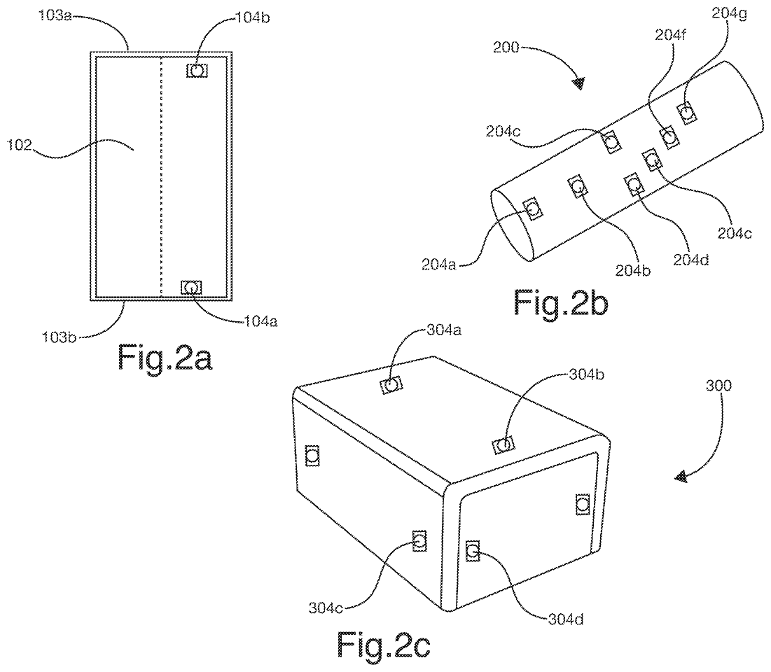

Sensors 104 in the form of data loggers (HOBO MX2201 Data Loggers, Onset Computer Corporation, Bourne, MA) were fitted onto 3 locations on each of 4 DonJoy UltraSling (DonJoy Performance, Dallas, Tex.) shoulder slings, which are routinely provided to patients postoperatively. As can be seen in FIG. 1 and also with reference to FIG. 2a, data loggers may be preferably situated at one or more preferred locations on or within a rehabilitation device. While generally throughout this specification, the term “sensor” and “logger” are used interchangeably because of the incorporation of a sensor into the data loggers used, it is to be understood that these two functions could also be separated, with a sensor separate from but in communications with a data logger. For experimentation, the sensors were manually sutured into 3 locations on each shoulder sling 102. For instance, a one-inch slit may be made in the center of the sling bolster's outer cloth. A padded sensor is placed in the foam of the bolster. The cloth is then closed with a running 2-0 nylon suture (Ethicon, Cincinnati, Ohio) and then covered with a padded Aquacel Silver dressing (Convatec, Oklahoma City, Okla.). The sensors are retrieved after six-week prescribed immobilization and data uploaded via Bluetooth and converted to Microsoft Excel tabulated data. Temperature is recorded by the sensors 104 every 15 minutes throughout the six-week postoperative period. A validated algorithm, as described more fully below, was utilized to approximate total wear time.

A first sensor 104a was situated at an inner aspect of the bolster 102a that may be considered to be generally in contact with or sufficiently proximate to a user's abdomen 12 during proper wear. A second sensor 104b was situated at an inner aspect of the sling 102 proximate a user's medial elbow. A third sensor 104c was situated at an inner aspect of the sling 102 to be positioned at the palmar surface of the carpometacarpal joint. These locations are thought to provide an accurate, measurable proxy for proper use or wear of the device and more stable (e.g., less volatile) data points throughout the wearing or use of the device. The second sensor 104b and third sensor 104c may be better visualized in FIG. 2a, which generally shows a sling substrate laid flat, to be folded along dotted line and stitched along an elbow end 103a generally leaving an open end 103b, the sensors 104 facing an interior cavity formed by the substrate and adapted to receive a human elbow and forearm.

FIGS. 2b and 2c show alternative rehabilitation devices, such as a substantially cylindrical foam roller 200 and yoga block 300, respectively. Such devices can include sensors 204, 304 mounted thereon or therein, similar to those discussed above, so as to detect representations of use of the devices. For instance, a static stretch may be prescribed by a treatment provider, such that a certain portion or portions of the devices are to be contacted by a human body for expected durations, and such contact is then monitored. On a roller 200, sensors 204 may be aligned longitudinally, such as sensors 204a and 204b, be aligned radially, such as sensors 204c and 204d, and/or may be aligned helically such, as sensors 204e, 204f, and 204g. On a yoga block 300 (which may be a generally parallelepiped solid foam or cork structure), sensors 304 may be placed at or in predetermined locations expected to be individually or simultaneously contacted by a portion of a human body during a prescribed regimen. Sensors may be aligned on a same side of the block 300, such as sensors 304a and 304b, on adjacent sides of the block 300, such as sensors 304c and 304d, and/or on opposite sides of the block 300.

Regarding the preferred sling embodiment, a trial was conducted with the retrofitted slings in which they were worn throughout the day and at night by volunteers to simulate the patient experience including removing and re-applying the sling to best emulate exercise sessions and other instances in which a patient might remove the sling (e.g., for personal hygiene). The 4 participants kept detailed timetables of actual sling wear time to compare with the wear time detected by the temperature sensors 104. The sling was worn in both winter and summer months. The data loggers were set to record temperature readings every 15 minutes to ensure that the memory capacity of the device was not exceeded during a period of about 3 months.

To evaluate for any statistically significant differences in shoulder sling wear time approximation among the 3 different sensor locations, 3 unpaired 2-sample t tests (Microsoft Excel Microsoft Corporation, Redmond, Wash.) were run to compare algorithm-generated time approximations among groups. This was done to analyze whether there was a statistically significant difference (P<0.05) in the time approximations registered by each sensor location on the arm.

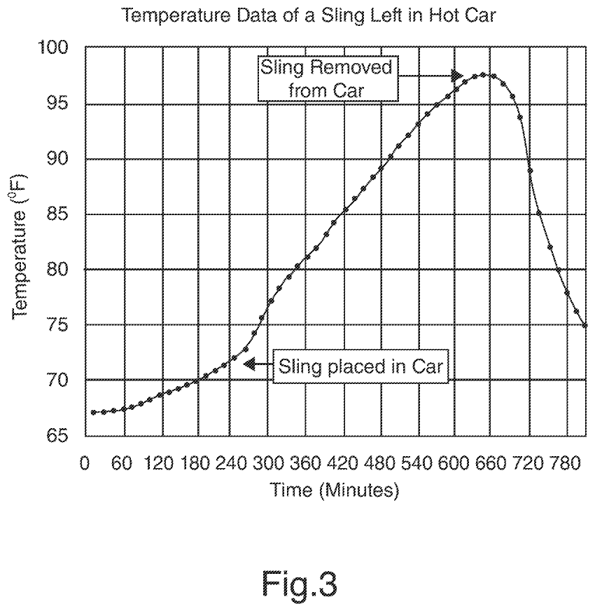

To provide an adequate proxy for use of a rehabilitative device, the algorithm for analyzing sensor data must account for differing sensed conditions. For instance, when a temperature sensor is being utilized, if the only data points reviewed are that of temperature, exclusive of any other information (e.g., time), then it is unlikely that accurate determinations of wear or use time can be calculated. As in, the analysis should account for a difference between body heat while the device is being worn or used and ambient heat in an environment. Specifically, what has been discovered is a difference between measurements taken and logged while wearing the shoulder sling retrofitted with the temperature sensors as compared to measurements take and logged while storing it in a hot environment. To confirm, the retrofitted sling was placed into the trunk of a car during a hot day with the outside temperatures ranging from 80 to 90° F. Afterward, the sensor data were evaluated for differences. The results can be seen in FIG. 3.

The ambient temperature control test indicated the effects of a hot environment on the data loggers. The loggers differentiated the ambient heat of the hot environment from body temperature using our algorithm. Although the final temperatures reached by the sensor in the hot environment versus when the sting was worn were similar, the final temperature was met at a much slower rate when the sensor was in a hot environment. When the sling was left in the trunk of a car with outside temperatures ranging between 80 and 90° F., it took 4.17 hours for the data logger to equilibrate to a temperature of 85° F. before eventually peaking at 97° F. approximately 6.83 hours later. In comparison, when the sling was actually worn, the temperature reached 85° F. in <45 minutes. Accordingly, the algorithm herein is able to discern the difference in heating and/or cooling rates to determine whether the shoulder sling is being worn. Implementation of the algorithm improves the accuracy of the loggers and decreases false positive readings. Accordingly, the sensors or loggers may either record a timestamp with each measurement or observation, or the time between measurements (i.e., sample rate) may be known and/or programmed into the logger(s).

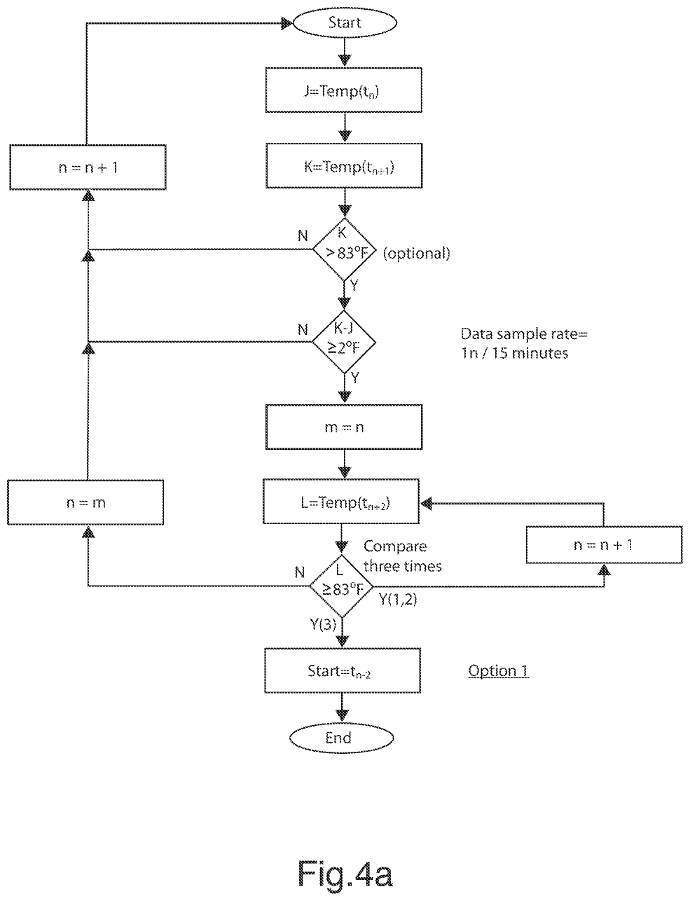

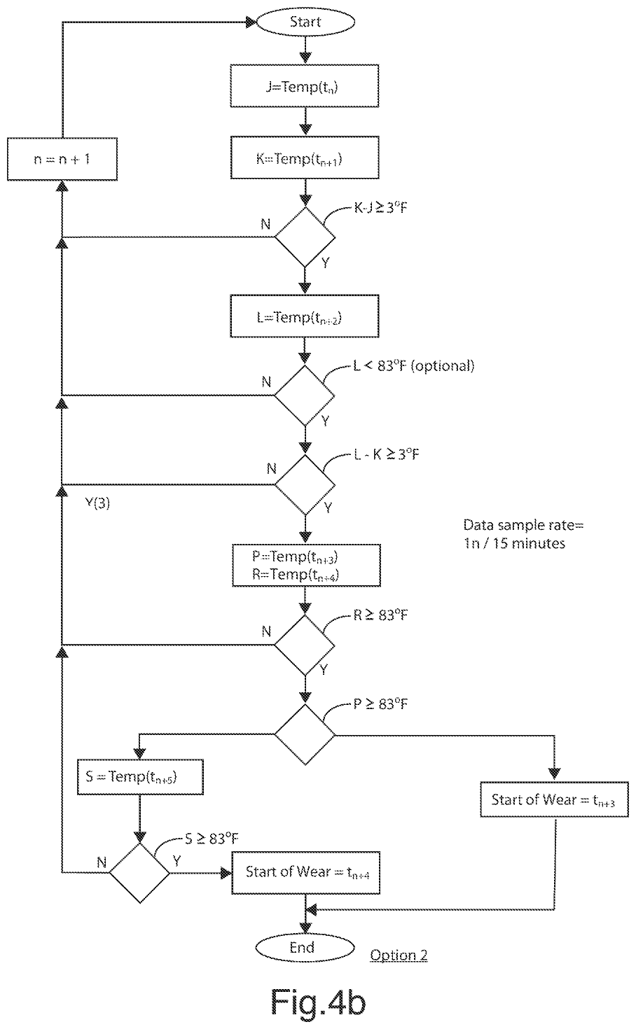

An algorithm has been developed to better approximate actual shoulder sling wear time based on information logged by the sensors 104 on the rehabilitative device 100. FIGS. 4a and 4b present flowcharts for the analytical determination, from logged data, of a start of a wear or use period, which can be subtracted from the end of wear or use time to determine a length of at least substantially continuous use of the device. For a given time point to be considered the start of a wear or use period (or “sling on” time), where the increments in “n” refer to subsequent measurements made at the predetermined sample rate (in this case 15 minutes), certain predetermined conditions must be met. These increments can be adjusted if a different sample rate is used, to analyze the requisite timing. First, either (Option 1, shown in FIG. 4a) the time point in question must be the first or second in a consecutive pair of points with a temperature increase of at least 2° F. immediately followed by a temperature of <83° F. or (Option 2, shown in FIG. 4b) the time point in question must be associated with the first in a consecutive pair of points with a temperature increase of at least 3° F. immediately followed by another consecutive pair of points with a temperature increase of at least 3° F. followed by a temperature≥83° F. within 30 minutes.

Second, after the analysis in Option 1 or Option 2, the temperature must remain ≥83° F. for at least 30 minutes. Third, the temperature cannot exceed 100° F. For instance, time to be considered the start of a wear period, either option 1 or option 2 must be true (where tₙ is the time point in question or sling on time):

```
Option 1:
      (Temp(tₙ₊₁) − Temp(tₙ)) ≥ 2°F
            ~and~
      Temp(tₙ₊₁) < 83°F (optional)
            ~and~
          Temp(tₙ₊₂) ≥ 83°F
            ~and~
          Temp(tₙ₊₃) ≥ 83°F
            ~and~
          Temp(tₙ₊₄) ≥ 83°F
Option 2:
      (Temp(tₙ₊₁) − Temp(tₙ)) ≥ 3°F
            ~and~
    (Temp(tₙ₊₂) − Temp(tₙ₊₁)) ≥ 3°F
            ~and~
      Temp(tₙ₊₂) < 83°F (optional)
            ~and~
  Temp(tₙ₊₃) ≥ 83°F ~OR~ Temp(tₙ₊₄) ≥ 83°F
            ~and~
          Temp(tₙ₊₅) ≥ 83°F
        ~and, if Temp(tₙ₊₃) < 83°F~
          Temp(tₙ₊₆) ≥ 83°F
```

Analysis may be aborted, or reset in the chronological review of logged data (n=n+1 to analyze next data points), if the measured characteristic (e.g., temperature) falls outside of a desired range, such as if the temperature exceeds 100° F. In that case, the algorithm will be searching for the next chronologically occurring instance of either option 1 or option 2, above, to flag a start of a wear period. Optionally, though the above options refer to tₙ as a preferred sling on time, the sling on time may be specified as the next time (tₙ₊₁) if tₙ and/or tₙ₊₁ were less than 83° F.

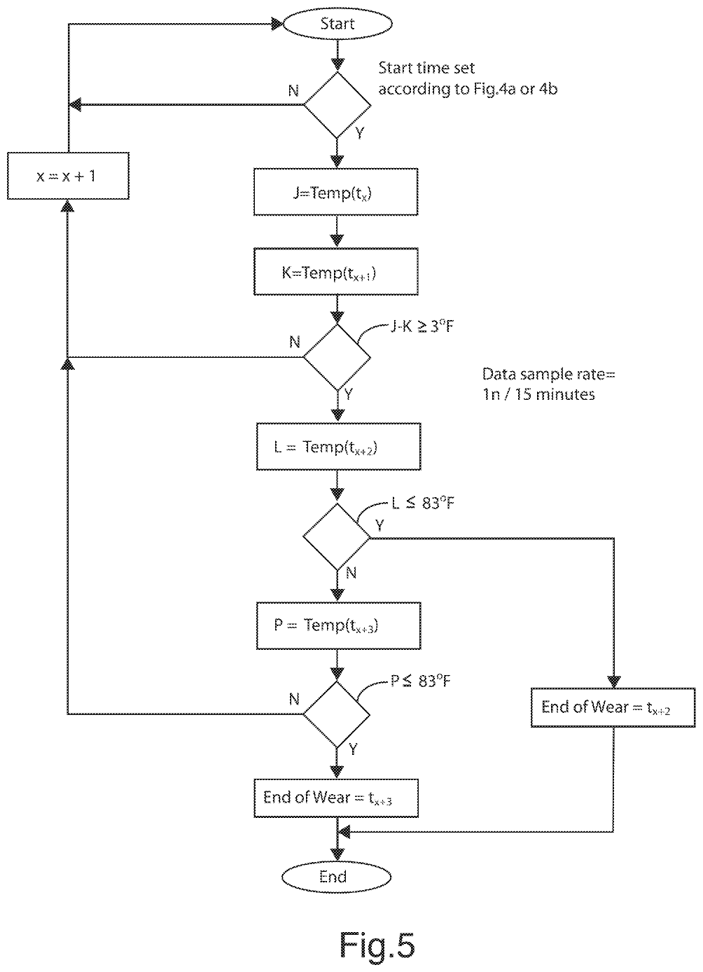

FIG. 5 presents a flowchart for the analytical determination, from logged data, of an end of a wear or use period. For a given time point (tₓ)to be considered the end of a wear period (or “sling off” time), the following conditions must be met. First, the time point in question must be part of an established wear period, or a start of wear period (or “sling on” time”) must have already occurred based on analysis of logged data, as described above. Second, the time point in question must be the first of a consecutive pair of time points with a temperature decrease of at least 3° F. Third, the temperature must decrease below 83° F. within 30 minutes of the 3° F. drop in temperature.

For time to be considered the end of a wear period, both condition 1 and condition 2 are preferably required, where the increments in “x” refer to subsequent measurements made at the predetermined sample rate (in this case 15 minutes). These increments can be adjusted if a different sample rate is used, to analyze the requisite timing.

(Temp(tₓ)−Temp(tₓ₊₁))≥3° F. Condition 1

˜and˜

(Temp(tₓ₊₂))≤83° F. ˜OR˜ Temp (tₓ₊₃))≤83° F. Condition 2

The algorithm can be programmed into instructions executable by a computer or other device with a microprocessor, such as by using a formula in Microsoft Excel to allow for automated analysis of data received or requested from the sensors/loggers 104. In this way, rehabilitative device wear or use time periods may be calculated (by subtracting the sling on time from the sling off time), recorded, charted and further compared to functional outcomes through physical testing of the wearer. Additionally or alternatively, adjustments may be made to prescribed therapeutic regimens or even the rehabilitative device, itself. For instance, the prescribed therapeutic regimen information may be changed to increase or decrease prescribed usage time of the device, or the device may be resized or exchanged for a different device, or one or more sensors may be activated and/or deactivated on the device to appropriately track usage with existing or modified regimen information.

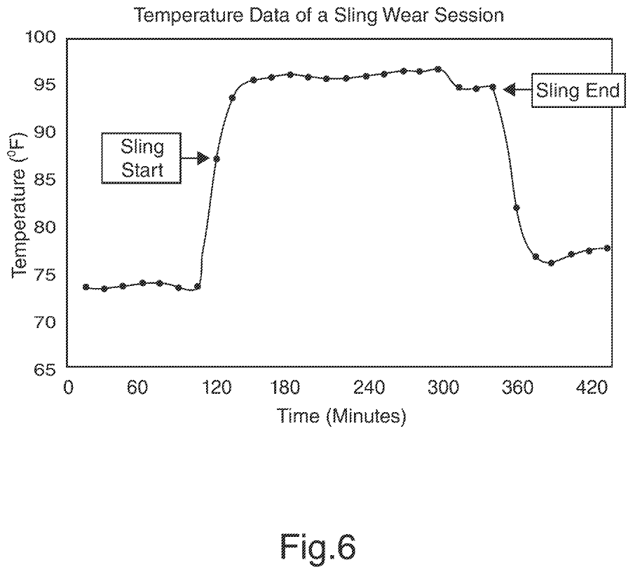

During experimentation actual wear time for the modified slings with data loggers was a total of 171.63 hours. The slings were monitored for approximately 1101 hours. Per the algorithm described above, the data loggers installed on the bolster, elbow, and wrist areas calculated a total estimated wear time of 167.75, 171.00, and 172.25 hours, respectively. The diagnostic accuracies for the bolster, elbow, and wrist areas of the data loggers were 99.5%, 99.1%, and 99.3%, respectively. True positive, true negative, false positive, false negative, sensitivity, specificity, positive predictive value, and negative predictive value are presented in Table 1. No statistically significant difference was shown in shoulder sling wear time approximation among the 3 data logger locations (P>0.05). FIG. 6 depicts a representative wear graph showing temperatures logged every 15 minutes of actual device usage. This graph shows an alternative sling on time determination, selecting from the first temperature that is greater than 83° F., rather than the first

Accordingly, the described protocol is an accurate method to measure or approximate (within incremental sample rate times) patient compliance with shoulder sling wear time by subtracting a start of wear period time from an end of wear period time. Although the placement of the data logger at the bolster location had the highest diagnostic accuracy of 99.5%, no significant difference was shown among the 3 locations tested and all locations had an accuracy>99%, and only a single location is sufficient.

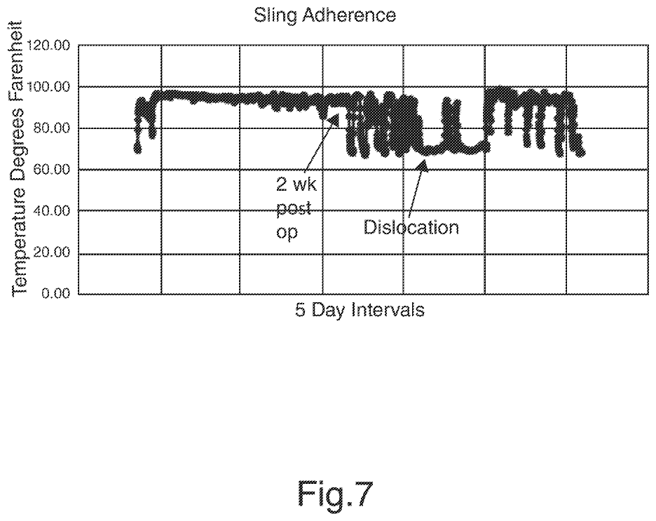

Turning now to FIG. 7 a sling adherence graph can be seen for a patient that underwent a left reverse shoulder replacement. As can be seen from the graph, the patient was very adherent with sling wear for two weeks then began to wear the sling much less and presented with a dislocated prosthesis at three weeks. The dislocation occurred during the third week postop during a stretch of days when the patient was not wearing the sling, at least as represented by the high variability of temperature measurements seen on the graph occurring after the two-week post-operative timeframe. The patient was thereafter treated with a closed reduction without a repeat operation. The patient wore the sling consistently at 90% adherence rate, as indicated by logged representations of wear (e.g., logged temperature measurements) for six weeks and had no further complication. At 6 months follow up, the patient's visual analog pain score (VAS) was 1 and the patient's American Shoulder and Elbow Surgeons Score (ASES) was 85 with forward elevation to 150 degrees, abduction to 100 degrees, external rotation to 30 degrees and internal rotation to the low back.

The foregoing is illustrative only of the principles of embodiments according to the present invention. Modifications and changes will readily occur to those skilled in the art, so it is not desired to limit the invention to the exact disclosure herein provided. While the preferred embodiment has been described, the details may be changed without departing from the invention, which is defined by the claims.

What is claimed is:

1. A device comprising:
a substrate in a form suitable for providing a rehabilitation of a portion of a human body;
one or more sensors capable of detecting a representation of a use of the substrate;
nonvolatile memory for storing detected representations as electronic data;
a communication system capable of transmitting the electronic data to an electronic device; and,
a power source suitable for powering the one or more sensors, the nonvolatile memory, and the communication system.

2. The device of claim 1, wherein the substrate comprises a shoulder sling.

3. The device of claim 2, wherein each of the one or more sensors is placed at a location selected from the group consisting of: an inner aspect of a bolster to be positioned between the shoulder sling and a human abdomen, a first inner aspect of the shoulder sling to be positioned proximate a human medial elbow, and a second inner aspect of the shoulder sling to be positioned proximate a human carpometacarpal joint palmar surface.

4. The device of claim 2, wherein the nonvolatile memory receives and maintains the electronic data directly or indirectly from the one or more sensors, where the detected representations are at least one selected from the group consisting of: temperature measurement, capacitance, light level, human pulse rate, and human pulse-oxygenation level.

5. The device of claim 4, wherein the communication system is capable of communicating the data to an external storage medium disposed in the electronic device.

6. The device of claim 5, wherein the communication system is capable of wireless communication with the electronic device.

7. The device of claim 5, wherein the communication system is configured to transmit data.

8. The device of claim 7, wherein the communication system is configured to transmit data automatically.

9. The device of claim 7, wherein the communication system is configured to transmit data only in response to a request from the electronic device.

10. The device of claim 5, wherein the electronic device is selected from the group consisting of a personal computer, a mobile phone, a tablet computer.

11. The device of claim 4, wherein the one or more sensors are capable of detecting a representation of a use of the substrate at a programmable sample rate.

12. The device of claim 1, wherein the rehabilitation device comprises a brace.

13. A method for monitoring use of a device comprising:
providing a rehabilitation device comprising one or more sensors;
logging data from the one or more sensors into nonvolatile memory, the data being representative time that the rehabilitation device is used by a human; and
comparing the logged data from the one or more sensors to at least one of data manually recorded by the human and first prescriptive use regimen data; and
archiving the logged data.

14. The method of claim 13, wherein the rehabilitation device comprises a sling.

15. The method of claim 13, wherein the rehabilitation device further comprises a communication system and wherein the logging step further comprises communicating the actual wearing time data to the communication system.

16. The method of claim 13, further comprising the step of:
transmitting the data to an external storage medium.

17. The method of claim 13, further comprising the step of physically altering the rehabilitation device.

18. The method of claim 13, wherein the one or more sensors comprise at least one of the following: temperature sensor, capacitance sensor, pulse-oxygenation sensor, heart rate sensor, or blood pressure sensor.

19. The method of claim 13, further comprising the step of generating a second prescriptive use regimen data that is different than the first prescriptive use regimen data.

20. The method of claim 13, wherein the rehabilitation device comprises a brace.
